(19) United States
(12) Patent Application Publication
(10) Pub. No.: US 2019/0298666 A1
(43) Pub. Date: Oct. 3, 2019

(54) PROMOTION OF T LYMPHOCYTE PROLIFERATION

(71) Applicant: Regents of the University of Minnesota, Minneapolis, MN (US)

(72) Inventors: David A. Potter, Minneapolis, MN (US); Michael Farrar, Minneapolis, MN (US); Zhijun Guo, Minneapolis, MN (US)

(21) Appl. No.: 16/281,929

(22) Filed: Feb. 21, 2019

Related U.S. Application Data
(60) Provisional application No. 62/633,467, filed on Feb. 21, 2018.

(51) Int. Cl. A61K 31/155; A61P 35/00; A61K 31/145; A61K 35/17; C12N 5/0783; C07K 16/28

(52) U.S. Cl. CPC A61K 31/155; A61P 35/00; A61K 31/145; C07K 16/2818; C12N 5/0636; C07K 16/2809; A61K 35/17

(57) ABSTRACT

Certain embodiments of the invention provide a method of promoting T lymphocyte proliferation, comprising contacting in vitro, ex vivo or in vivo a population of cells comprising T lymphocytes with an effective amount of N1-hexyl-N5-benzyl biguanide (HBB), or a pharmaceutically acceptable salt thereof, wherein contact with HBB, or a pharmaceutically acceptable salt thereof, causes proliferation of the T lymphocytes.

### RELATED APPLICATION

This application claims the benefit of priority of U.S. Provisional Application Ser. No. 62/633,467 filed on Feb. 21, 2018, which application is incorporated by reference herein.

### GOVERNMENT FUNDING

This invention was made with government support under R01-CA113570 awarded by the National Cancer Institute. The government has certain rights in the invention.

### BACKGROUND OF THE INVENTION

Immunotherapy helps to repair, stimulate, or enhance the body's natural immune responses and may be used to treat a variety of diseases, including cancer and certain autoimmune diseases. Immunotherapies may use substances made by the body or in a laboratory to improve or restore immune system function. Cancer immunotherapy approaches include, e.g., active immunization, reversal of immunosuppression, nonspecific immune stimulation and adoptive cell transfer (ACT). To date, ACT has been demonstrated to be an effective form of immunotherapy for cancer treatment and has achieved promising results in cancer clinical trials (Rosenberg et al., Nature Reviews Cancer, 8:299-308 (2008)). However, ACT often includes the modification and expansion of immune cells (e.g., T lymphocytes) in vitro or ex vivo. Current methods for immune cell expansion have certain drawbacks. For example, PMA and ionomycin may be used to induce T cell activation; however, these agents can result in significant cell death. Other methods have involved the use of concanavalin A and CD3/CD28 beads, but do not result in CD8 cells of sufficient activity or viability (J. Immunol. 128, 337; J. Immunother. 27, 405. Palacios, R., 1982). T cell expansion methods using IL-2, IL-7 and IL-15 have also been reported; however, the cost of such cytokines is high (Clin Exp Immunol. 2005 November; 142(2):292-302; Clinical & Translational Immunology (2015) 4, e31; doi:10.1038/cti.2014.31; Journal of Immunological Methods 417 (2015) 134-138). Furthermore, while IL-7 and IL-2 can also be used to promote the proliferation or death of CD4+ T cells by modulating Fas expression, there can be susceptibility to FasL associated cell death making this method variable in success (J Immunol 2003; 171:61-8).

Thus, there is a need for new methods and compositions for use in immunotherapies (e.g., for the treatment cancer).

### SUMMARY OF THE INVENTION

Accordingly, as described herein, N1-hexyl-N5-benzyl-biguanide (HBB) can be used to expand T cells (e.g., CD4+ and CD8+), e.g., in combination with CD3/CD28 antibody stimulation. HBB can also simultaneously inhibit tumor growth and metastasis by inhibiting intratumoral EET biosynthesis (J Clin Invest. 2012 January; 122(1):178-91). EETs have low affinity receptors so it is possible to inhibit cancer cell intrinsic EETs while at the same time stimulating CD8+ effector T cell lymphocyte growth. HBB is unique in having both properties simultaneously. Additionally, HBB stimulation does not require PMA and ionomycin stimulation, which can result in significant cell death. The cost of HBB as a small biguanide molecule is also likely to be less than PMA and ionomycin or cytokines, including IL-7 and IL-2.

Accordingly, certain embodiments of the invention provide a method of promoting T lymphocyte proliferation, comprising contacting a population of cells comprising T lymphocytes with an effective amount of N1-hexyl-N5-benzyl biguanide (HBB), or a pharmaceutically acceptable salt thereof, wherein contact with HBB, or a pharmaceutically acceptable salt thereof, causes proliferation of the T lymphocytes. In certain embodiments, the proliferated T lymphocytes are not regulatory T cells (Tregs).

Certain embodiments of the invention also provide a method of selecting patients for treating a disease or disorder, comprising:

a) contacting a patient sample comprising T lymphocytes ex-vivo with an effective amount of N1-hexyl-N5-benzyl biguanide (HBB), or a pharmaceutically acceptable salt thereof, under conditions suitable to promote T lymphocyte proliferation;

b) measuring the population of T lymphocytes before and after contacting with HBB; and

c) administering the population of proliferated T lymphocytes to the patient if the population has increased by at least about 20%. In certain embodiments, the proliferated T lymphocytes are not regulatory T cells.

Certain embodiments of the invention provide a method of treating a disease or disorder in a patient, comprising:

a) contacting a patient sample comprising T lymphocytes with an effective amount of N1-hexyl-N5-benzyl biguanide (HBB), or a pharmaceutically acceptable salt thereof, under conditions suitable to promote T lymphocyte proliferation, wherein contact with HBB, or a pharmaceutically acceptable salt thereof, generates a population of proliferated T lymphocytes; and

b) administering the population of proliferated T lymphocytes to the patient. In certain embodiments, the proliferated T lymphocytes are not regulatory T cells.

Certain embodiments of the invention provide, a method of promoting T lymphocyte proliferation in a patient in need thereof, comprising administering to the patient an effective amount of N1-hexyl-N5-benzyl biguanide (HBB), or a pharmaceutically acceptable salt thereof. In certain embodiments, the proliferated T lymphocytes are not regulatory T cells.

Certain embodiments of the invention provide a method of activating the immune system in a patient in need thereof, comprising administering to the patient an effective amount of N1-hexyl-N5-benzyl biguanide (HBB), or a pharmaceutically acceptable salt thereof.

Certain embodiments of the invention provide a method of treating an autoimmune disease in a patient comprising administering to the patient an effective amount of N1-hexyl-N5-benzyl biguanide (HBB), or a pharmaceutically acceptable salt thereof.

Certain embodiments of the invention provide N1-hexyl-N5-benzyl biguanide (HBB), or pharmaceutically acceptable salt thereof, for use in promoting T lymphocyte proliferation in vivo.

Certain embodiments of the invention provide the use of N1-hexyl-N5-benzyl biguanide (HBB), or a pharmaceutically acceptable salt thereof, to prepare a medicament for promoting T lymphocyte proliferation in vivo.

Certain embodiments of the invention provide N1-hexyl-N5-benzyl biguanide (HBB), or pharmaceutically acceptable salt thereof, for use in activating the immune system.

Certain embodiments of the invention provide the use of N1-hexyl-N5-benzyl biguanide (HBB), or a pharmaceutically acceptable salt thereof, to prepare a medicament for activating the immune system in a patient.

Certain embodiments of the invention provide N1-hexyl-N5-benzyl biguanide (HBB), or pharmaceutically acceptable salt thereof, for use in the prophylactic or therapeutic treatment of an autoimmune disease.

Certain embodiments of the invention provide the use of N1-hexyl-N5-benzyl biguanide (HBB), or a pharmaceutically acceptable salt thereof, to prepare a medicament for treating an autoimmune disease in a patient.

Certain embodiments of the invention provide a method of inhibiting the generation of regulatory T cells in a cell population comprising contacting the cell population with an effective amount of N1-hexyl-N5-benzyl biguanide (HBB), or a pharmaceutically acceptable salt thereof.

Certain embodiments of the invention also provide a method of inhibiting the generation of regulatory T cells in a patient in need thereof comprising administering to the patient an effective amount of N1-hexyl-N5-benzyl biguanide (HBB), or a pharmaceutically acceptable salt thereof.

Certain embodiments of the invention provide a method for treating cancer in a patient comprising administering to the patient an effective amount of 1) N1-hexyl-N5-benzyl biguanide (HBB), or a pharmaceutically acceptable salt thereof; and 2) one or more immune checkpoint inhibitors, wherein the one or more immune checkpoint inhibitors are selected from the group consisting of CTLA4, PD-1 and PD-L1 inhibitors.

Certain embodiments of the invention provide N1-hexyl-N5-benzyl biguanide (HBB), or pharmaceutically acceptable salt thereof, for use in the prophylactic or therapeutic treatment of a cancer, in combination with one or more immune checkpoint inhibitors, wherein the one or more immune checkpoint inhibitors are selected from the group consisting of CTLA4, PD-1 and PD-L1 inhibitors.

Certain embodiments of the invention provide the use of N1-hexyl-N5-benzyl biguanide (HBB), or a pharmaceutically acceptable salt thereof, to prepare a medicament for treating cancer in a patient, in combination with one or more immune checkpoint inhibitors, wherein the one or more immune checkpoint inhibitors are selected from the group consisting of CTLA4, PD-1 and PD-L1 inhibitors.

Certain embodiments of the invention provide a pharmaceutical composition comprising N1-hexyl-N5-benzyl biguanide (HBB), or pharmaceutically acceptable salt thereof, and one or more immune checkpoint inhibitors, wherein the one or more immune checkpoint inhibitors are selected from the group consisting of CTLA4, PD-1 and PD-L1 inhibitors.

Certain embodiments of the invention provide a kit comprising N1-hexyl-N5-benzyl biguanide (HBB), or a pharmaceutically acceptable salt thereof, and one or more immune checkpoint inhibitors, packaging material, and instructions for administering HBB, or a pharmaceutically acceptable salt thereof, and the inhibitor to a patient to treat cancer, wherein the one or more immune checkpoint inhibitors are selected from the group consisting of CTLA4, PD-1 and PD-L1 inhibitors.

Certain embodiments of the invention provide a method of inhibiting mechanistic target of rapamycin (mTOR), electron transfer (ETC) and/or oxygen consumption rate (OCR) in a cell, comprising contacting the cell with an effective amount of N1-hexyl-N5-benzyl biguanide (HBB), or a pharmaceutically acceptable salt thereof, wherein contact with HBB, or a pharmaceutically acceptable salt thereof, causes inhibition of mTOR, ETC and/or OCR.

Certain embodiments of the invention provide a method of inhibiting mechanistic target of rapamycin (mTOR) and/or electron transfer (ETC) in a cell, comprising contacting the cell with an effective amount of N1-hexyl-N5-benzyl biguanide (HBB), or a pharmaceutically acceptable salt thereof, wherein contact with HBB, or a pharmaceutically acceptable salt thereof, causes inhibition of mTOR, ETC and/or OCR.

Certain embodiments of the invention provide a method of inhibiting mechanistic target of rapamycin (mTOR), electron transfer (ETC) and/or oxygen consumption rate (OCR) in one or more cells in a patient in need thereof, comprising administering to the patient an effective amount of N1-hexyl-N5-benzyl biguanide (HBB), or a pharmaceutically acceptable salt thereof.

Certain embodiments of the invention provide a method of inhibiting mechanistic target of rapamycin (mTOR) and/or electron transfer (ETC) in one or more cells in a patient in need thereof, comprising administering to the patient an effective amount of N1-hexyl-N5-benzyl biguanide (HBB), or a pharmaceutically acceptable salt thereof.

### BRIEF DESCRIPTION OF THE FIGURES

FIGS. 1A-B. HBB induces the growth of CD4 and CD8 mouse splenic T cells at concentrations that inhibit growth of the MCF-7 breast cancer cells. FIGS. 1A-B. Freshly harvested C57BL/6 splenocytes were seeded at 80,000 cells/well in a 96-well plate coated with anti-mouse CD3 (10 ug/ml) and anti-mouse CD28 (1 ug/ml) antibodies. After 24 hour incubation with HBB (0-50 uM), cells were then labeled with anti-mouse CD4-eFlour450, anti-mouse CD8 FITC and anti-mouse Thy1.2 APC-eFluor780 antibodies and analyzed by a flowcytometer. Thy1.2 positive single lymphocytes were gated as T cells and CD4 positive and CD8 positive cells were counted. FIG. 1A. Results are expressed as mean±S.D. (n=6). FIG. 1B. MCF-7 breast cancer cells were incubated in the same concentration range of HBB, also for 24 hours. MTT assay of cell numbers.

FIGS. 2A-C. Effect of HBB on mouse CD4 and CD8 cells at 24 hours.

FIG. 3. Effect of HBB on CYP2J2-mediated EET biosynthesis.

FIG. 4. Freshly harvested C57BL/6 splenocytes were seeded at 80,000 cells/well in a 96-well plate coated with anti-mouse CD3 (10 ug/ml) and anti-mouse CD28 (1 ug/ml) antibodies. After 24 hour incubation with metformin (0-10 mM), cells were then labeled with anti-mouse CD4-eFlour450, anti-mouse CD8 FITC and anti-mouse Thy1.2 APC-eFluor780 antibodies and analyzed by a flowcytometer. Thy1.2 positive single lymphocytes were gated as T cells and CD4 positive and CD8 positive cells were counted.

FIG. 5. Cytochrome P450 expression analysis in human T cells by PCR. Total mRNAs were extracted from CD4, CD8 and NK cells from healthy human donors and reverse transcribed into cDNA libraries. Specific CYP primers were used for PCR amplification and PCR products were resolved on a 1.5% agrose gel. A normal human liver cDNA library was used as a positive control. Sizes of all CYP PCR products agree with theoretical values. Among the four CYPs tested, all are expressed in high level in human liver as expected and only CYP2J2 is expressed at detectable level in CD4, CD8 and NK cells. Representative gel image of PCR products of CD8 cells from one donor is shown. Primers used are as follow: CYP2C8 forward: 5′-AGATCAGAATTTTCTCACCC-3′(SEQ ID NO:1), reverse: 5′-AACTTCGTGTAAGAGCAACA-3′ (SEQ ID NO:2); CYP2J2 forward: 5′-GCCCGGGAGTCCATGCCCTA-3′ (SEQ ID NO:3), reverse: 5′-GGGCAGGTGGTACCCAGCCA-3′ (SEQ ID NO:4); CYP3A4 forward: 5′-GCCTGGTGCTCCTCTATCTA-3′ (SEQ ID NO:5), reverse: 5′-GGCTGTTGACCATCATAAAAG-3′ (SEQ ID NO:6); CYP4A11 forward: 5′-CATGGCAGACTCTGTACGAGTG-3′ (SEQ ID NO:7), reverse: 5′-CTGATGGCTGAAGGCACACTTC-3′ (SEQ ID NO:8). Sizes of amplicons of the PCR reaction are 158, 126, 187 and 113 for CYP2C8, 2J2, 3A4 and 2J2, respectively.

FIGS. 6A-6C. HBB blocks the IL2-dependent conversion of CD4+CD25+FOXP3− and CD4+CD25−FOXP3+ Treg progenitor populations into Tregs in the presence of HBB.

FIGS. 7A-D. HBB inhibits the differentiation of naive T cells into Tregs in the presence of IL2 and TGFb.

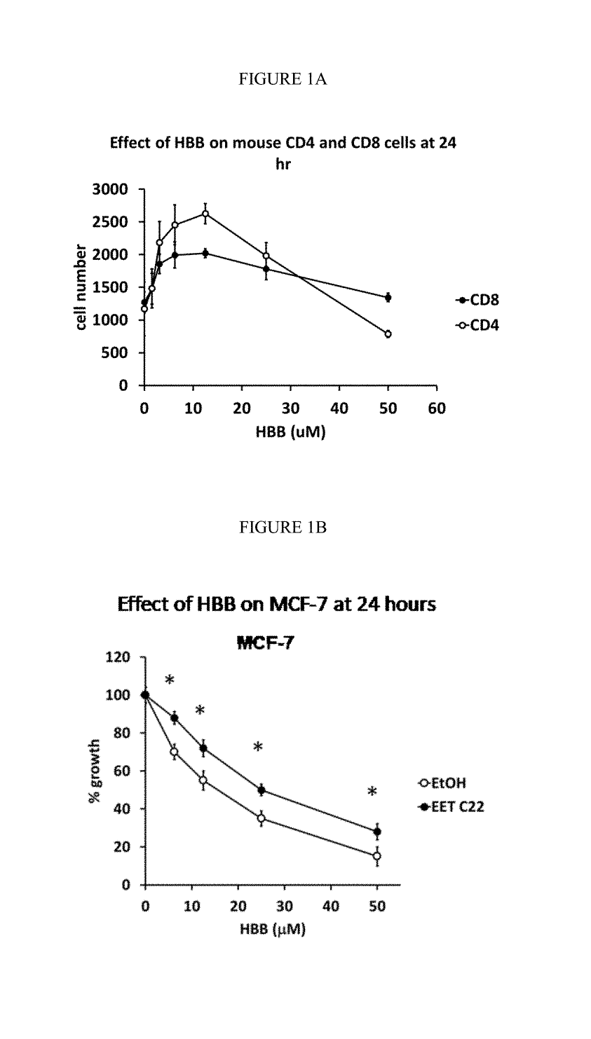

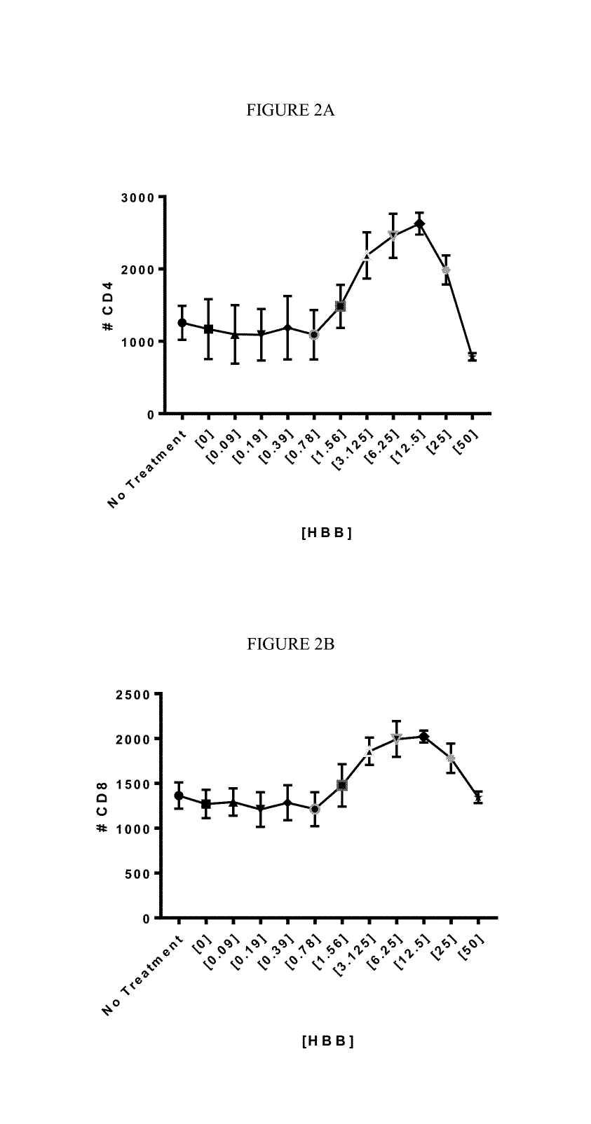

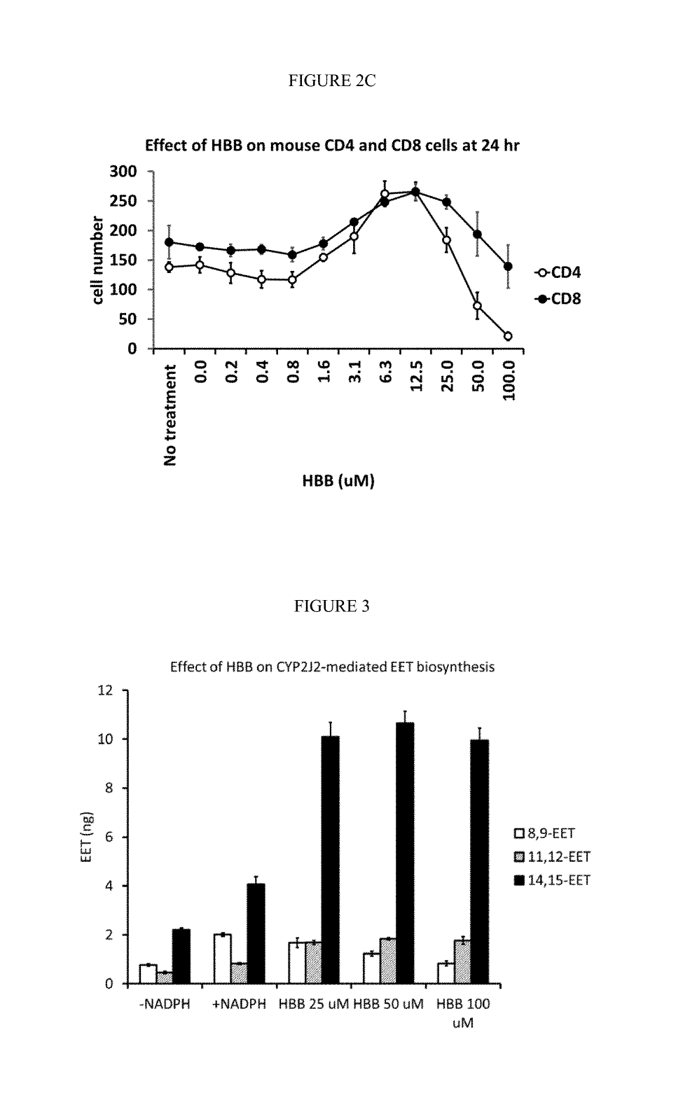

### DETAILED DESCRIPTION

As described herein, N1-hexyl-N5-benzyl biguanide (HBB) is capable of promoting T lymphocyte proliferation (e.g., CD4+ and/or CD8+ T cells). Accordingly, HBB may be used in a variety of treatment methods for a range of disease and disorders. For example, HBB may be used to, e.g., promote the in vitro or ex vivo expansion of T cells for immunotherapy (e.g., cancer immunotherapy); expand CD4+T helper cells and/or CAR-T cells for the treatment of graft vs. host disease or other autoimmune diseases; expand cytotoxic CD8+ lymphocytes or CAR-T cells for the treatment of cancer (e.g., hematopoietic or solid tumors); or promote endogenous cytotoxic CD8 lymphocytes for treatment of a malignancy (e.g., breast cancer).

As described herein, HBB has also been shown to inhibit tumor growth. Specifically, HBB has been shown to potently and specifically inhibit CYP3A4 arachidonic acid (AA) epoxygenase activity in tumors, which causes an inhibition of oxygen consumption rates (OCR), electron transfer chain (ETC) and tumor growth. The differing effects of HBB on cancer cells versus lymphocytes is related to the absence of the CYP3A4 target and the presence of CYP2J2 in lymphocytes, in contrast to tumor cells (e.g., breast), where CYP3A4 is present (see, FIGS. 3 and 5 and Siest et al., Drug Metabolism and Dispostion 36:182-189, discussing CYP expression is lymphocytes). Thus, the technology described herein solves the problem of simultaneously using a biguanide to inhibit an epithelial malignancy, such as breast cancer, by blocking oxidative phosphorylation (OXPHOS) (Cell Chemical Biology. 2017 Oct. 19; 24(10) 1259-1275), while at the same time causing cytotoxic CD8 lymphocytes to proliferate and attack a tumor. The ability of HBB to both inhibit tumor growth and promote T cell proliferation is distinct from certain other anti-cancer agents. For example, cyclophosphamide is known to inhibit tumor cells such as breast cancer cells, but also inhibits lymphocyte proliferation. While metformin has been demonstrated to support the function of leukocytes in animals (Proc Natl Acad Sci USA. 2015 Feb. 10; 112(6):1809-14), it is unknown whether this effect is direct or indirect. Furthermore, metformin exhibits activity against breast cancer cells only in the 5 mM concentration range while HBB exhibits activity in the 20 micromolar range (Cell Chemical Biology. 2017 Oct. 19; 24(10) 1259-1275, which is incorporated by reference in its entirety for all purposes).

### Methods of Activating the Immune System

Accordingly, certain embodiments of the invention provide a method of activating the immune system in a patient in need thereof, comprising administering to the patient an effective amount of N1-hexyl-N5-benzyl biguanide (HBB), or a pharmaceutically acceptable salt thereof.

N1-hexyl-N5-benzyl biguanide (HBB):

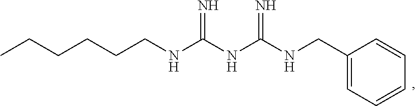 [SMILES: CCCCCCNC(=N)NC(=N)NCc1ccccc1]

or pharmaceutically acceptable salt thereof, may be prepared according to methods described in US patent application publication US2015-0342909, which is incorporated by reference in its entirety for all purposes.

As used herein, the term “activating the immune system” refers to the proliferation of T cells (e.g., cytotoxic CD8+ effector T cells or certain CD4+ T cells).

Thus, certain embodiments of the invention also provide a method of promoting T lymphocyte proliferation, comprising contacting a population of cells comprising T lymphocytes with an effective amount of N1-hexyl-N5-benzyl biguanide (HBB), or a pharmaceutically acceptable salt thereof, wherein contact with HBB, or a pharmaceutically acceptable salt thereof, causes proliferation of the T lymphocytes.

As used herein, the term “proliferation” refers to an increase in the number of cells, as a result of cell growth and cell division. Thus, as described herein, HBB may be used to increase the number T lymphocytes in a cell population, as compared to a control (e.g., the number of T lymphocytes in a cell population not contacted with HBB).

As used herein, the terms “T lymphocyte” and “T cell” refer to lymphocytes that comprise a T cell receptor. Examples of T lymphocytes, include, but are not limited to, effector T cells, T helper cells, cytotoxic T cells, adoptive T cells, memory T cells, natural killer T cells, mucosal associated invariant T cells and gamma delta T cells, as well as T cells comprising chimeric antigen receptors (CARs or CAR-T). In certain embodiments, a T cell may express certain glycoproteins on its surface, such as CD4 (i.e., CD4+ T cell) and/or CD8 (i.e., CD8+ T cell). In certain embodiments, the T cell is a T cell other than a regulatory T cell.

Thus, in certain embodiments, the proliferated T lymphocytes are selected from the group consisting of effector T cells, T helper cells, cytotoxic T cells, adoptive T cells, memory T cells, natural killer T cells, mucosal associated invariant T cells, gamma delta T cells and chimeric antigen receptor T cells (CAR-T).

In certain embodiments, the proliferated T lymphocytes comprise CD4+ T cells. In certain embodiments, the proliferated T lymphocytes comprise CD4+ T helper cells.

As described herein, HBB inhibits the generation of regulatory T cells. Thus, in certain embodiments, HBB does not promote regulatory T cell proliferation and the proliferated T lymphocytes are not regulatory T cells. In certain embodiments, the proliferated T lymphocytes are not CD4+ regulatory T cells. In certain embodiments, the T lymphocytes are not CD25+ regulatory T cells. In certain embodiments, the T lymphocytes are not forkhead box P3 positive (FOXP3+) regulatory T cells. In certain embodiments, the T lymphocytes are not CD4+FOXP3+CD25+ regulatory T cells.

In certain embodiments, the proliferated T lymphocytes comprise CD8+ T cells. In certain embodiments, the proliferated T lymphocytes comprise cytotoxic CD8+ lymphocytes. For example, cytotoxic CD8+ T cells may be used to inhibit the growth of cancer. By stabilizing tumor specific CD8+ T cells in vivo, it may be possible to promote their activity against cancer (Blood. 2006 Feb. 15; 107(4):1325-31.; Cancer Immunol Res. 2017 January; 5(1):9-16).

In certain embodiments, the proliferated T lymphocytes comprise adoptive CD8+ T cells. For example, adoptive CD8+ T cells could be used to promote cancer vaccine activity (Clin Cancer Res. 2014 Mar. 1; 20(5):1355-65).

In certain embodiments, the proliferated T lymphocytes comprise CAR-T cells.

In certain embodiments, the proliferated T lymphocytes do not express CYP3A4. In certain embodiments, the proliferated T lymphocytes express CYP2J2. CYP2J2 synthesizes arachidonic acid epoxides called EETs which promote hematopoietic lineage cell expansion (Nature. 2015 Jul. 23; 523(7561):468-71).

In certain embodiments, the number of T lymphocytes in the population of cells increases by, e.g., about 5%, 10%, 20%, 30%, 40%, 50%, 60%, 70%, 80%, 90%, 95%, 99%, 100%, 200%, or more after being contacted with HBB, or a pharmaceutically acceptable salt thereof.

In certain embodiments, the population of cells comprising T lymphocytes is contacted with HBB, or a pharmaceutically acceptable salt thereof, in vitro, ex vivo or in vivo.

In certain embodiments, the population of cells comprising T lymphocytes is contacted with HBB, or a pharmaceutically acceptable salt thereof, in vitro. For example, cultured T lymphocytes or modified T lymphocytes (e.g., CAR-T cells) may be contacted with HBB in vitro. In certain embodiments, the method further comprises administering the HBB-treated, proliferated T lymphocytes to a patient.

In certain embodiments, the population of cells comprising T lymphocytes is contacted with HBB, or a pharmaceutically acceptable salt thereof, ex vivo. For example, a biological sample comprising a population of cells comprising T lymphocytes may be obtained from a patient and subsequently contacted with HBB, or a pharmaceutically acceptable salt thereof. Methods of obtaining such samples are known in the art (e.g., from a blood sample, bone marrow sample or a tissue sample (e.g., thymus or spleen)). Accordingly, certain embodiments of the invention provide a method of promoting T lymphocyte proliferation, comprising contacting a population of cells comprising T lymphocytes obtained from a patient, with an effective amount of N1-hexyl-N5-benzyl biguanide (HBB), or a pharmaceutically acceptable salt thereof, wherein contact with HBB, or a pharmaceutically acceptable salt thereof, causes proliferation of the T lymphocytes (e.g., proliferation of T lymphocytes other than regulatory T cells). In certain embodiments, the method further comprises administering the HBB-treated, proliferated T lymphocytes to the patient.

Thus, certain embodiments of the invention provide a method of promoting T lymphocyte proliferation, comprising:

Certain embodiments of the invention provide a method of selecting patients for treating a disease or disorder, comprising:

a) contacting a patient sample comprising T lymphocytes ex-vivo with an effective amount of N1-hexyl-N5-benzyl biguanide (HBB), or a pharmaceutically acceptable salt thereof, under conditions suitable to promote T lymphocyte proliferation;

b) measuring the population of T lymphocytes before and after contacting with HBB; and

c) administering the population of proliferated T lymphocytes to the patient if the population has increased by at least about 20%. In certain embodiments, the population of proliferated T lymphocytes is administered to the patient if the population increased by at least about 25%, 30%, 35%, 40%, 45%, 50%, 55%, 60%, 65%, 70%, 75%, 80%, 85%, 90%, 95%, or more. In certain embodiments, the proliferated T lymphocytes are not regulatory T cells.

Certain embodiments of the invention also provide a method of treating a disease or disorder in a patient, comprising:

a) contacting a patient sample comprising T lymphocytes with an effective amount of N1-hexyl-N5-benzyl biguanide (HBB), or a pharmaceutically acceptable salt thereof, under conditions suitable to promote T lymphocyte proliferation, wherein contact with HBB, or a pharmaceutically acceptable salt thereof, generates a population of proliferated T lymphocytes; and

b) administering the population of proliferated T lymphocytes to the patient. In certain embodiments, the proliferated T lymphocytes are not regulatory T cells.

Certain embodiments of the invention provide a method of treating a disease or disorder in a patient, comprising:

a) obtaining a biological sample from the patient, wherein the sample comprises a population of cells comprising T lymphocytes;

b) contacting the sample with an effective amount of N1-hexyl-N5-benzyl biguanide (HBB), or a pharmaceutically acceptable salt thereof, under conditions suitable to promote T lymphocyte proliferation, wherein contact with HBB, or a pharmaceutically acceptable salt thereof, generates a population of proliferated T lymphocytes; and

c) administering the population of proliferated T lymphocytes to the patient. In certain embodiments, the proliferated T lymphocytes are not regulatory T cells.

In certain embodiments, the disease or disorder is cancer or an autoimmune disease. In certain embodiments, the disease or disorder is cancer. In certain embodiments, the cancer is a type of cancer type described herein. In certain embodiments, the disease or disorder is an autoimmune disease (e.g., an autoimmune disease described herein).

In certain embodiments, a method described herein further comprises activating the T lymphocytes. In certain embodiments, the T lymphocytes are activated prior to, simultaneously with, or subsequent to contact with HBB, or a pharmaceutically acceptable salt thereof. Methods of activating T lymphocytes are known in the art. For example, T lymphocytes may be activated by contacting the cells with anti-CD3 and/or anti-CD28 antibodies. Thus, in certain embodiments, the method further comprises contacting the population of cells comprising T lymphocytes with an anti-CD3 and/or anti-CD28 antibody.

In certain embodiments, the population of cells comprising T lymphocytes is contacted with HBB, or a pharmaceutically acceptable salt thereof, in vivo. Thus, certain embodiments of the invention provide a method of promoting T lymphocyte proliferation in a patient in need thereof, comprising administering an effective amount of N1-hexyl-N5-benzyl biguanide (HBB), or a pharmaceutically acceptable salt thereof, to the patient, wherein contact with HBB, or a pharmaceutically acceptable salt thereof, causes proliferation of the T lymphocytes. In certain embodiments, the proliferated T lymphocytes are not regulatory T cells.

Certain embodiments of the invention also provide a method of treating an autoimmune disease in a patient comprising administering to the patient an effective amount of N1-hexyl-N5-benzyl biguanide (HBB), or a pharmaceutically acceptable salt thereof.

Certain embodiments of the invention provide a method of treating cancer in a patient in need thereof, comprising administering to the patient an effective amount of N1-hexyl-N5-benzyl biguanide (HBB), or a pharmaceutically acceptable salt thereof. In certain embodiments, the cancer is a cancer other than breast cancer. In certain embodiments, the cancer does not comprise and/or was not determined to comprise increased expression of at least one CYP and/or decreased expression of soluble epoxide hydrolase (EPHX2) (e.g., HBB, or a pharmaceutically acceptable salt thereof, is administered to promote T lymphocyte proliferation and not to directly inhibit tumor growth).

In certain embodiments, the methods further comprise administering to the patient one or more additional therapeutic agents.

In certain embodiments, the one or more additional therapeutic agents are useful for treating cancer (e.g., a chemotherapeutic agent, immunotherapeutic agent, hormonal agent or radiation therapy).

In certain embodiments, the one or more additional therapeutic agents are a chemotherapeutic agent. In certain embodiments, the chemotherapeutic agent is selected from tamoxifen, fulvestrant, raloxifene, anastrozole, letrozole, exemestane, paclitaxel, docetaxel, ixabepilone, eribulin, capecitabine, gemcitabine, vinorelbine, palbociclib, everolimus, trastuzumab, pertuzumab, lapatinib and other HER2 receptor tyrosine kinase inhibitors.

In certain embodiments, the one or more additional agents activate the immune system. In certain embodiments, the one or more additional agent is an immune checkpoint inhibitor antibody, or fragment thereof, such as an anti-CTLA4, anti PD-1 or anti PD-L1 antibody.

In certain embodiments, the one or more additional therapeutic agents are useful for treating an autoimmune disease or disorder. In certain embodiments, the additional therapeutic agent is an IL-6 antibody, such as toxilizumab. In certain embodiments, the additional therapeutic agent is a TNF-alpha antibody, such as, e.g., infliximab or adalimumab.

The one or more additional therapeutic agents may be administered either simultaneously or sequentially with the original therapeutic (i.e., HBB, or a pharmaceutically acceptable salt thereof, or the proliferated T lymphocytes). In certain embodiments, the one or more additional therapeutic agents are administered simultaneously with the original therapeutic. In certain embodiments, a composition (e.g., a pharmaceutical composition) comprising the original therapeutic, and the one or more additional therapeutic agents are administered. In certain embodiments, the original therapeutic and the one or more additional therapeutic agents are administered sequentially. In certain embodiments, the original therapeutic is administered first and the one or more additional therapeutic agents are administered second. In certain embodiments, the one or more additional therapeutic agents are administered first and the original therapeutic is administered second.

### Methods of Inhibiting the Generation of Regulatory T Cells

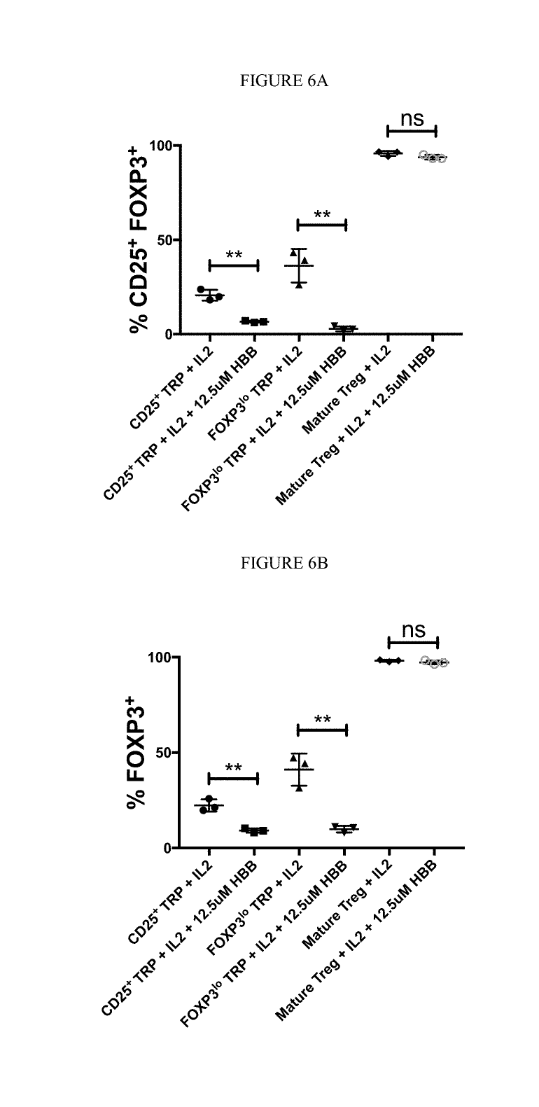

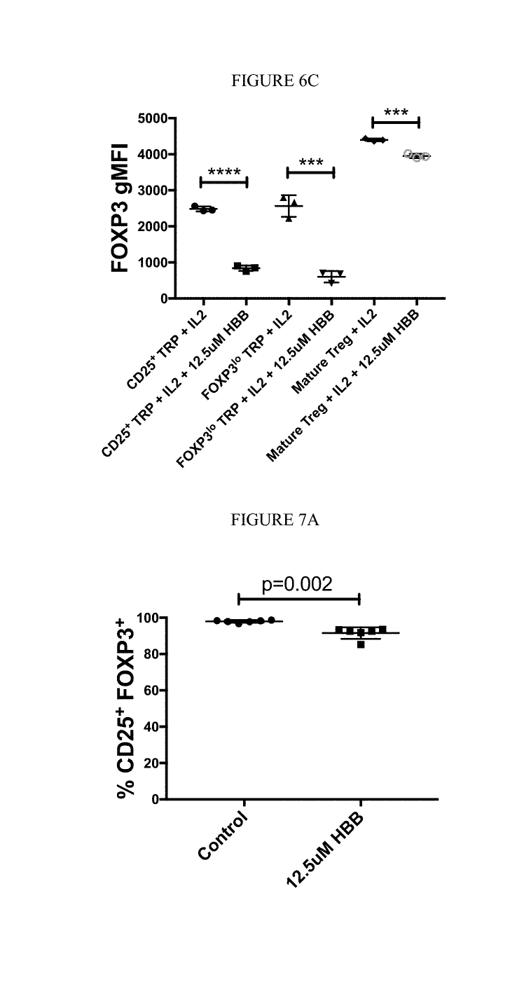

As described herein, HBB inhibits the conversion of progenitor regulatory T cells into regulatory T cells and can also inhibit the differentiation of naive T cells into regulatory T cells (see, FIGS. 6A-6C; and 7A-7D).

Accordingly, certain embodiments of the invention provide a method of inhibiting the generation of regulatory T cells in a cell population comprising contacting the cell population with an effective amount of N1-hexyl-N5-benzyl biguanide (HBB), or a pharmaceutically acceptable salt thereof. In certain embodiments, the contacting occurs in vitro, ex vivo or in vivo. In certain embodiments, the cell population comprises progenitor regulatory T cells. In certain embodiments, the cell population comprises naive T cells. In certain embodiments, the cell population comprises both progenitor regulatory T cells and naive T cells.

Certain embodiments of the invention also provide a method of inhibiting the generation of regulatory T cells in a patient in need thereof comprising administering to the patient an effective amount of N1-hexyl-N5-benzyl biguanide (HBB), or a pharmaceutically acceptable salt thereof.

As used herein the term “generation of regulatory T cells” may be used to refer to, e.g., regulatory T cell maturation, conversion of progenitor regulatory T cells into regulatory T cells, or the differentiation of naive T cells into regulatory T cells. Thus, in certain embodiments, HBB, or a pharmaceutically acceptable salt thereof, inhibits regulatory T cell maturation. In certain embodiments, HBB, or a pharmaceutically acceptable salt thereof, inhibits the conversion of progenitor regulatory T cells into regulatory T cells. In certain embodiments, HBB, or a pharmaceutically acceptable salt thereof, inhibits the differentiation of naive T cells into regulatory T cells.

In certain embodiments, the progenitor regulatory T cell is CD25+. In certain embodiments, the progenitor regulatory T cell is CD25−. In certain embodiments, the progenitor regulatory T cell is FOXP3−. In certain embodiments, the progenitor regulatory T cell is FOXP3+. In certain embodiments, the progenitor regulatory T cell is CD4+CD25+FOXP3−. In certain embodiments, the progenitor regulatory T cell is CD4+CD25−FOXP3+.

In certain embodiments, the naive T cell is a CD4+, non-regulatory T cell.

In certain embodiments, a regulatory T cell is CD25+FOXP3+.

In certain embodiments the generation of regulatory T cells is inhibited by at least about 15%, 20%, 25%, 30%, 35%, 40%, 45%, 50%, 55%, 60%, 65%, 70%, 75%, 80%, 85%, 90%, 95%, or more as compared to a control (e.g., a population of cells or a patient not contacted with HBB).

In certain embodiments, the patient has cancer (e.g., a cancer described herein). In certain embodiments, the cancer patient has an increased abundance of regulatory T cells (e.g., as compared to a control or reference value). In certain embodiments, the cancer patient has an increased abundance of regulatory T cells in the tumor microenvironment (e.g., as compared to a control or reference value).

Certain embodiments of the invention also provide a method of selecting a cancer patient for treatment, comprising:

a) measuring the abundance of regulatory T cells in a sample obtained from the cancer patient; and

b) administering an effective amount of N1-hexyl-N5-benzyl biguanide (HBB), or a pharmaceutically acceptable salt thereof, if the abundance of regulatory T cells in the sample is increased as compared to a reference or control value. In certain embodiments the abundance of regulatory T cells is increased by at least about 15%, 20%, 25%, 30%, 35%, 40%, 45%, 50%, 55%, 60%, 65%, 70%, 75%, 80%, 85%, 90%, 95%, or more as compared to a reference or control value. In some embodiments, the reference value is the abundance of regulatory T cells in a reference sample. In some embodiments, the reference value may be obtained from a plurality of samples or a population of subjects. The abundance of regulatory T cells may be measured using assays known in the art (e.g., through the use of flow cytometry).

In certain embodiments, the sample is a tumor sample. In certain embodiments, the sample is a blood sample or other fluid comprising T cells.

HBB in combination with Certain Immune Checkpoint Inhibitors

As described herein, HBB may be used to enhance the efficacy of certain immune checkpoint therapies. While not wishing to be bound by theory, it is believed that HBB may enhance PD-1 and PD-L1 antibody therapy of cancer by selectively inhibiting tumor epithelial oxygen and glucose consumption while maintaining effector T cell and other immunocyte function. HBB may also enhance CTLA4 inhibitors, either alone or in combination with a PD-1 or PD-L1 inhibitor.

Accordingly, certain embodiments of the invention provide a method for treating cancer in a patient comprising administering an effective amount of 1) N1-hexyl-N5-benzyl biguanide (HBB), or a pharmaceutically acceptable salt thereof; and 2) one or more immune checkpoint inhibitors to the patient, wherein the immune checkpoint inhibitors are selected from PD-1, PD-L1 and CTLA4 inhibitors.

“Immune checkpoint inhibitors” are agents that block certain proteins made by some types of immune system cells, such as T cells, and some cancer cells. These proteins help keep immune responses in check and can keep T cells from killing cancer cells. When these proteins are blocked, the “brakes” on the immune system are released and T cells are able to kill cancer cells better. Examples of checkpoint proteins found on cells or cancer cells include PD-1, PD-L1 and CTLA-4.

PD-1 is a 288 amino acid protein receptor expressed on activated T-cells and B-cells, natural killer cells and monocytes. PD-1 is a member of the CD28/CTLA-4 (cytotoxic T lymphocyte antigen)/ICOS (inducible co-stimulator) family of T-cell co-inhibitory receptors (Chen et al. 2013, Nat. Rev. Immunol. 13: 227-242). The protein has an extracellular N-terminal domain which is IgV-like, a transmembrane domain and an intracellular domain containing an immunoreceptor tyrosine-based inhibitory (ITIM) motif and an immunoreceptor tyrosine-based switch (ITSM) motif (Chattopadhyay et al 2009, Immunol. Rev.). The primary function of PD-1 is to attenuate the immune response (Riley 2009, Immunol. Rev. 229:114-125). PD-1 has two ligands, PD-Ligand1 (PD-L1) and PD-L2. PD-L1 (CD274, B7H 1) is expressed widely on both lymphoid and non-lymphoid tissues such as CD4 and CD8 T-cells, macrophage lineage cells, peripheral tissues as well as on tumor cells, virally-infected cells and autoimmune tissue cells. PD-L2 (CD273, B7-DC) has a more restricted expression than PD-L1, being expressed on activated dendritic cells and macrophages (Dong et al 1999, Nature Med.). PD-1 binding to its ligands results in decreased T-cell proliferation and cytokine secretion, compromising humoral and cellular immune responses.

CTLA4 or CTLA-4 (cytotoxic T-lymphocyte-associated protein 4), also known as CD152 (cluster of differentiation 152), is a protein receptor that, functioning as an immune checkpoint, downregulates immune responses. CTLA4 is constitutively expressed in regulatory T cells but only upregulated in conventional T cells after activation—a phenomenon which is particularly notable in cancers. It acts as an “off” switch when bound to CD80 or CD86 on the surface of antigen-presenting cells. The CTLA-4 protein is encoded by the CTLA4 gene in humans.

The terms “CTLA4 inhibitor” and “PD-1 or PD-L1 inhibitor” as used herein includes any compound or treatment capable of inhibiting the expression and/function of CTLA4, PD-1 or PD-L1 (e.g., inhibits transcription, RNA maturation, RNA translation, post-translational modification, or function of the protein). For example, in certain embodiments, the inhibitor detectably inhibits the expression level or biological activity of CTLA4, PD-1 or PD-L1. In certain embodiments, the inhibitor inhibits the expression level or biological activity of the protein by at least about 5%, at least about 10%, at least about 20%, at least about 30%, at least about 40%, at least about 50%, at least about 60%, at least about 70%, at least about 80%, or at least about 90%.

The inhibitor may be of natural or synthetic origin. For example, it may be a nucleic acid, a polypeptide, a protein, a peptide, or an organic compound. In one embodiment, the inhibitor is an siRNA, shRNA, a small molecule or an antibody.

In certain embodiments, the inhibitor is a polypeptide, for example, an antibody against CTLA4, PD-1 or PD-L1, or a fragment or derivative thereof, such as a Fab fragment, a CDR region, or a single chain antibody.

The term “small molecule” includes organic molecules having a molecular weight of less than about 1000 amu. In one embodiment a small molecule can have a molecular weight of less than about 800 amu. In another embodiment a small molecule can have a molecular weight of less than about 500 amu.

In certain embodiments, the inhibitor is a PD-1 inhibitor. PD-1 inhibitors, are known in the art. For example, PD-1 inhibitors include, but are limited to, nivolumab, pembrolizumab, pidilizumab, AMP-224, MEDI0680 and spartalizumab. Accordingly, in certain embodiments, the PD-1 inhibitor is an anti-PD-1 antibody, or a fragment thereof. In certain embodiments, the PD-1 inhibitor is nivolumab, pembrolizumab, pidilizumab, AMP-224, MEDI0680 or spartalizumab.

In certain embodiments, the inhibitor is a PD-L1 inhibitor. PD-L1 inhibitors, are known in the art. For example, PD-L1 inhibitors include, but are limited to, atezolizumab, avelumab, durvalumab and BMS-936559. Thus, in certain embodiments, the PD-L1 inhibitor is an anti-PD-L1 antibody, or a fragment thereof. In certain embodiments, the PD-L1 inhibitor is atezolizumab, avelumab, durvalumab or BMS-936559.

In certain embodiments, the inhibitor is a CTLA4 inhibitor. CTLA4 inhibitors, are known in the art. For example, CTLA4 inhibitors include, but are limited to ipilimumab and tremelimumab. Accordingly, in certain embodiments, the CTLA4 inhibitor is an anti-CTLA4 antibody, or a fragment thereof. In certain embodiments, the CTLA4 inhibitor is ipilimumab or tremelimumab.

HBB and the one or more immune checkpoint inhibitors may be administered either simultaneously or sequentially. In certain embodiments, HBB is administered simultaneously with the inhibitor. In certain embodiments, a composition (e.g., a pharmaceutical composition) comprising HBB and the inhibitor is administered. In certain embodiments, HBB and the inhibitor are administered sequentially. In certain embodiments, the HBB is administered first and the inhibitor is administered second. In certain embodiments, the inhibitor is administered first and HBB is administered second.

Certain embodiments of the invention provide N1-hexyl-N5-benzyl biguanide (HBB), or a pharmaceutically acceptable salt thereof, for the prophylactic or therapeutic treatment of a cancer, in combination with one or more immune checkpoint inhibitors, wherein the immune checkpoint inhibitors are selected from PD-1, PD-L1 and CTLA4 inhibitors.

As used herein, the term “in combination with” refers to the simultaneous or sequential use of HBB, or a pharmaceutically acceptable salt thereof, and one or immune checkpoint inhibitors, as well as compositions comprising HBB, or a pharmaceutically acceptable salt thereof, and one or more immune checkpoint inhibitors.

Certain embodiments of the invention provide the use of N1-hexyl-N5-benzyl biguanide (HBB), or a pharmaceutically acceptable salt thereof, to prepare a medicament for treating cancer in a patient (e.g. a patient such as a human), in combination with one or more immune checkpoint inhibitors, wherein the immune checkpoint inhibitors are selected from PD-1, PD-L1 and CTLA4 inhibitors.

Certain embodiments of the invention provide N1-hexyl-N5-benzyl biguanide (HBB), or a pharmaceutically acceptable salt thereof, for medical therapy, in combination with one or more immune checkpoint inhibitors, wherein the immune checkpoint inhibitors are selected from PD-1, PD-L1 and CTLA4 inhibitors.

Certain embodiments of the invention provide a pharmaceutical composition comprising N1-hexyl-N5-benzyl biguanide (HBB), or pharmaceutically acceptable salt thereof, and one or more immune checkpoint inhibitors, wherein the immune checkpoint inhibitors are selected from PD-1, PD-L1 and CTLA4 inhibitors.

Certain embodiments of the invention provide a pharmaceutical composition comprising N1-hexyl-N5-benzyl biguanide (HBB), or a pharmaceutically acceptable salt thereof, and one or more immune checkpoint inhibitors, for the prophylactic or therapeutic treatment of cancer, wherein the immune checkpoint inhibitors are selected from PD-1, PD-L1 and CTLA4 inhibitors.

Certain embodiments of the invention provide a kit comprising N1-hexyl-N5-benzyl biguanide (HBB), or a pharmaceutically acceptable salt thereof, and one or more immune checkpoint inhibitors, packaging material, and instructions for administering HBB, or a pharmaceutically acceptable salt thereof, and the inhibitor to a patient to treat cancer, wherein the immune checkpoint inhibitors are selected from PD-1, PD-L1 and CTLA4 inhibitors.

In certain embodiments, the cancer is a cancer described herein.

mTOR inhibition and HBB in Combination with mTOR Inhibitors

Certain embodiments of the invention also provide a method of inhibiting a mechanistic target of rapamycin (mTOR) in a cell, comprising contacting the cell with an effective amount of N1-hexyl-N5-benzyl biguanide (HBB), or a pharmaceutically acceptable salt thereof, wherein contact with HBB, or a pharmaceutically acceptable salt thereof, causes inhibition of mTOR.

In certain embodiments, the cell is contacted with HBB, or a pharmaceutically acceptable salt thereof, in vitro, ex vivo or in vivo.

Certain embodiments of the invention provide a method of inhibiting a mechanistic target of rapamycin (mTOR) in a patient in need thereof, comprising administering an effective amount of N1-hexyl-N5-benzyl biguanide (HBB), or a pharmaceutically acceptable salt thereof, to the patient.

Certain embodiments of the invention also provide a method for treating cancer in a patient comprising administering an effective amount of 1) N1-hexyl-N5-benzyl biguanide (HBB), or a pharmaceutically acceptable salt thereof; and 2) a mechanistic target of rapamycin (mTOR) inhibitor to the patient.

The term “mTOR inhibitor” as used herein includes any compound or treatment capable of inhibiting the expression and/function of mTOR (e.g., inhibits transcription, RNA maturation, RNA translation, post-translational modification, or function of the protein). For example, in certain embodiments, the inhibitor detectably inhibits the expression level or biological activity of mTOR. In certain embodiments, the inhibitor inhibits the expression level or biological activity of the protein by at least about 5%, at least about 10%, at least about 20%, at least about 30%, at least about 40%, at least about 50%, at least about 60%, at least about 70%, at least about 80%, or at least about 90%.

The inhibitor may be of natural or synthetic origin. For example, it may be a nucleic acid, a polypeptide, a protein, a peptide, or an organic compound. In one embodiment, the inhibitor is an siRNA, shRNA, a small molecule or an antibody.

In certain embodiments, the inhibitor is a polypeptide, for example, an antibody against mTOR, or a fragment or derivative thereof, such as a Fab fragment, a CDR region, or a single chain antibody.

The term “small molecule” includes organic molecules having a molecular weight of less than about 1000 amu. In one embodiment a small molecule can have a molecular weight of less than about 800 amu. In another embodiment a small molecule can have a molecular weight of less than about 500 amu.

mTOR inhibitors, are known in the art. For example, mTOR inhibitors include, but are limited to rapamycin, rapalogs (e.g., sirolimus, temsirolimus, everolimus and ridaforolimus) and MLN0128 (TAK228). Thus, in certain embodiments, the mTOR inhibitor is rapamycin, a rapalog (e.g., sirolimus, temsirolimus, everolimus or ridaforolimus) or MLN0128 (TAK228). In certain embodiments, the mTOR inhibitor targets mTORC1. In certain embodiments, the mTOR inhibitor targets both mTORC1 and mTORC2.

HBB and the mTOR inhibitor may be administered either simultaneously or sequentially. In certain embodiments, HBB is administered simultaneously with the inhibitor. In certain embodiments, a composition (e.g., a pharmaceutical composition) comprising HBB and the inhibitor is administered. In certain embodiments, HBB and the inhibitor are administered sequentially. In certain embodiments, the HBB is administered first and the inhibitor is administered second. In certain embodiments, the inhibitor is administered first and HBB is administered second.

Certain embodiments of the invention provide N1-hexyl-N5-benzyl biguanide (HBB), or a pharmaceutically acceptable salt thereof, for the prophylactic or therapeutic treatment of a cancer, in combination with a mTOR inhibitor.

As used herein, the term “in combination with” refers to the simultaneous or sequential use of HBB, or a pharmaceutically acceptable salt thereof, and a mTOR inhibitor, as well as compositions comprising HBB, or a pharmaceutically acceptable salt thereof, and a mTOR inhibitor.

Certain embodiments of the invention provide the use of N1-hexyl-N5-benzyl biguanide (HBB), or a pharmaceutically acceptable salt thereof, to prepare a medicament for treating cancer in a patient (e.g. a patient such as a human), in combination with a mTOR inhibitor.

Certain embodiments of the invention provide N1-hexyl-N5-benzyl biguanide (HBB), or a pharmaceutically acceptable salt thereof, for medical therapy, in combination with a mTOR inhibitor.

Certain embodiments of the invention provide a pharmaceutical composition comprising N1-hexyl-N5-benzyl biguanide (HBB), or pharmaceutically acceptable salt thereof, and a mTOR inhibitor.

Certain embodiments of the invention provide a pharmaceutical composition comprising N1-hexyl-N5-benzyl biguanide (HBB), or a pharmaceutically acceptable salt thereof, and a mTOR inhibitor for the prophylactic or therapeutic treatment of cancer.

Certain embodiments of the invention provide a kit comprising N1-hexyl-N5-benzyl biguanide (HBB), or a pharmaceutically acceptable salt thereof, and a mTOR inhibitor, packaging material, and instructions for administering HBB, or a pharmaceutically acceptable salt thereof, and the inhibitor to a patient to treat cancer.

In certain embodiments, the cancer is a cancer described herein.

### Certain Other Embodiments

Certain embodiments of the invention also provide a method of suppressing electron transfer (ETC) in a cell (e.g., in the mitochondria of the cell), comprising contacting the cell with an effective amount of N1-hexyl-N5-benzyl biguanide (HBB), or a pharmaceutically acceptable salt thereof, wherein contact with HBB, or a pharmaceutically acceptable salt thereof, causes suppression of ETC. In certain embodiments, the cell is contacted with HBB, or a pharmaceutically acceptable salt thereof, in vitro, ex vivo or in vivo.

Certain embodiments of the invention provide a method of suppressing electron transfer (ETC) in a patient in need thereof, comprising administering an effective amount of N1-hexyl-N5-benzyl biguanide (HBB), or a pharmaceutically acceptable salt thereof, to the patient.

Certain embodiments of the invention also provide a method of inhibiting oxygen consumption rate (OCR) in a cell, comprising contacting the cell with an effective amount of N1-hexyl-N5-benzyl biguanide (HBB), or a pharmaceutically acceptable salt thereof, wherein contact with HBB, or a pharmaceutically acceptable salt thereof, causes inhibition of OCR. In certain embodiments, the cell is contacted with HBB, or a pharmaceutically acceptable salt thereof, in vitro, ex vivo or in vivo. In certain embodiments, the cell is not a breast cancer cell.

Certain embodiments of the invention provide a method of inhibiting oxygen consumption rate (OCR) in a patient in need thereof, comprising administering an effective amount of N1-hexyl-N5-benzyl biguanide (HBB), or a pharmaceutically acceptable salt thereof, to the patient. In certain embodiments, the patient does not have breast cancer.

### Diseases and Disorders

As described herein, methods of the invention may be used to treat cancer or a patient having cancer.

The terms “cancer” and “cancerous” refer to or describe the physiological condition in mammals that is typically characterized by unregulated cell growth and proliferation. Examples of cancer include, but are not limited to, carcinoma, lymphoma (e.g., Hodgkin's and non-Hodgkin's lymphoma), blastoma, sarcoma, and leukemia. More particular examples of cancers include squamous cell cancer, small-cell lung cancer, non-small cell lung cancer, adenocarcinoma of the lung, squamous carcinoma of the lung, cancer of the peritoneum, hepatocellular cancer, renal cell carcinoma, gastrointestinal cancer, gastric cancer, esophageal cancer, pancreatic cancer, glioma, cervical cancer, ovarian cancer, liver cancer, bladder cancer, hepatoma, breast cancer (e.g., endocrine resistant breast cancer), colon cancer, rectal cancer, lung cancer, endometrial or uterine carcinoma, salivary gland carcinoma, kidney cancer, liver cancer, prostate cancer, vulval cancer, thyroid cancer, hepatic carcinoma, melanoma, leukemia and other lymphoproliferative disorders, and various types of head and neck cancer.

Thus, in certain embodiments, the cancer is selected from the group consisting of carcinoma, lymphoma (e.g., Hodgkin's and non-Hodgkin's lymphoma), blastoma, sarcoma, and leukemia. In certain embodiments, the cancer is selected from the group consisting of squamous cell cancer, small-cell lung cancer, non-small cell lung cancer, adenocarcinoma of the lung, squamous carcinoma of the lung, cancer of the peritoneum, hepatocellular cancer, renal cell carcinoma, gastrointestinal cancer, gastric cancer, esophageal cancer, pancreatic cancer, glioma, cervical cancer, ovarian cancer, liver cancer, bladder cancer, hepatoma, breast cancer (e.g., endocrine resistant breast cancer), colon cancer, rectal cancer, lung cancer, endometrial or uterine carcinoma, salivary gland carcinoma, kidney cancer, liver cancer, prostate cancer, vulval cancer, thyroid cancer, hepatic carcinoma, melanoma, leukemia and other lymphoproliferative disorders, and various types of head and neck cancer. In certain embodiments, the cancer is breast cancer. In certain embodiments, the breast cancer is ER+. In certain embodiments, the breast cancer is HER2+. In certain embodiments, the breast cancer is triple negative breast cancer (ER−, PR− and HER2−). In certain embodiments, the breast cancer is estrogen positive HER2 negative breast cancer (ER+ HER2−). In certain embodiments, the breast cancer is ER− breast cancer. In certain embodiments, the breast cancer is metaplastic breast cancer. In certain embodiments, the breast cancer is androgen receptor positive breast cancer. In certain embodiments, the cancer patient has an increased abundance of regulatory T cells as compared to a control or reference value.

In certain embodiments, the disease or disorder is a disease or disorder other than cancer (e.g., certain in vivo embodiments). In certain embodiments, the cancer is a cancer other than breast cancer. In certain embodiments, the cancer does not comprise and/or was not determined to comprise increased expression of at least one CYP and/or decreased expression of soluble epoxide hydrolase (EPHX2) (e.g., HBB, or a pharmaceutically acceptable salt thereof, is administered to promote T lymphocyte proliferation or to inhibit regulatory T cell generation and not to directly inhibit tumor growth).

As described herein, methods of the invention may also be used to treat an autoimmune disease or a patient having an autoimmune disease.

In certain embodiments, the disease or disorder is an autoimmune disease. In certain embodiments, the patient has graft v. host disease.

The term “treat”, “treatment” or “treating,” to the extent it relates to a disease or condition includes inhibiting the disease or condition, eliminating the disease or condition, and/or relieving one or more symptoms of the disease or condition.

The term “patient” as used herein refers to any animal including mammals such as humans, higher non-human primates, rodents domestic and farm animals such as cow, horses, dogs and cats. In one embodiment, the patient is a human patient.

The phrase “effective amount” means an amount of a compound described herein that (i) increases the proliferation of T lymphocytes; (ii) treats or prevents the particular disease, condition, or disorder; (iii) attenuates, ameliorates, or eliminates one or more symptoms of the particular disease, condition, or disorder; or (iv) prevents or delays the onset of one or more symptoms of the particular disease, condition, or disorder described herein.

The terms “obtaining a sample from a patient”, “obtained from a patient” and similar phrasing, is used to refer to obtaining the sample directly from the patient, as well as obtaining the sample indirectly from the patient through an intermediary individual (e.g., obtaining the sample from a courier who obtained the sample from a nurse who obtained the sample from the patient).

### Administration

Biologically active agents described herein (e.g., HBB or proliferated T lymphocytes), can be formulated as a pharmaceutical compositions and administered to a mammalian host, such as a human patient in a variety of forms adapted to the chosen route of administration, i.e., orally or parenterally, by intravenous, intramuscular, topical or subcutaneous routes.

Thus, the compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of HBB, or other biologically active agents, can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The compounds may be conveniently formulated in a unit dosage form. In one embodiment, the invention provides a compound described herein formulated in such a unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

HBB can also be administered in combination with other therapeutic agents, for example, other agents that are useful for treating cancer or an autoimmune disease. Examples of such agents are described herein. Accordingly, one embodiment the invention also provides for the use of a composition comprising HBB, or a pharmaceutically acceptable salt thereof, at least one other therapeutic agent, and a pharmaceutically acceptable diluent or carrier. The invention also provides a kit comprising HBB, or a pharmaceutically acceptable salt thereof, at least one other therapeutic agent, packaging material, and instructions for administering HBB, and the other therapeutic agent or agents to an animal to treat cancer or an autoimmune disease.

The invention will now be illustrated by the following non-limiting Examples.

### Example 1. N1-hexyl-N5-benzyl-biguanide Promotes Proliferation of CD4+ and CD8+ T Lymphocytes

Solid tumors are often resistant to immunotherapy due to a hostile microenvironment for immune cells, characterized by hypoxia and low nutrients. Metformin has been shown to potentiate PD-1 blockade and improve intratumoral T-cell function and tumor response through reduction of hypoxia (Cancer Immunol Res. 2017 Jan. 1; 5(1) 9-16). A novel biguanide (neo-biguanide) that potentiates immunotherapy similar to metformin, but is more potent, would be more ideal for combination with immunotherapy. Recently, it was discovered that metformin binds to the mitochondria associated enzyme CYP3A4 and thereby suppresses the electron transport chain (ETC) and oxygen consumption rates (OCR) (Cell Chemical Biology. 2017 Oct. 19; 24(10) 1259-1275). Structural biology approaches led to discovery of N1-hexyl-N5-benzyl-biguanide (HBB), which binds to the CYP3A4 heme with higher affinity than metformin and much more potently inhibits the ETC and OCR (Cell Chemical Biology. 2017 Oct. 19; 24(10) 1259-1275). HBB potently and specifically inhibits CYP3A4 arachidonic acid (AA) epoxygenase activity, causing OCR and growth inhibition of breast cancer cell lines and established mouse mammary tumors (IC₅₀=3-30 uM). Because lymphocytes express relatively lower levels of the CYP3A4 target of HBB (Drug Metab Dispos. 2008 January; 36(1):182-9, which is incorporated by reference herein), it was hypothesized that HBB may permit T lymphocyte growth at concentrations that inhibit tumor epithelia. Therefore, effects of HBB on T cell proliferation and activation were tested with mouse splenocytes as described below.

### Methods

HBB effects on T cell growth were tested across a range of concentrations (0 to 50 uM). C57BL/6J mouse splenocytes were incubated with or without biguanides in the presence of anti-mouse CD3 and anti-mouse CD28 antibodies. After 24 hours, cells were labeled with fluorescently tagged antibodies to mouse Thy-1, CD4, CD8, CD25, CD44, CD62L and CD73 surface antigens and T cell subsets were analyzed by flow cytometry.

### Results

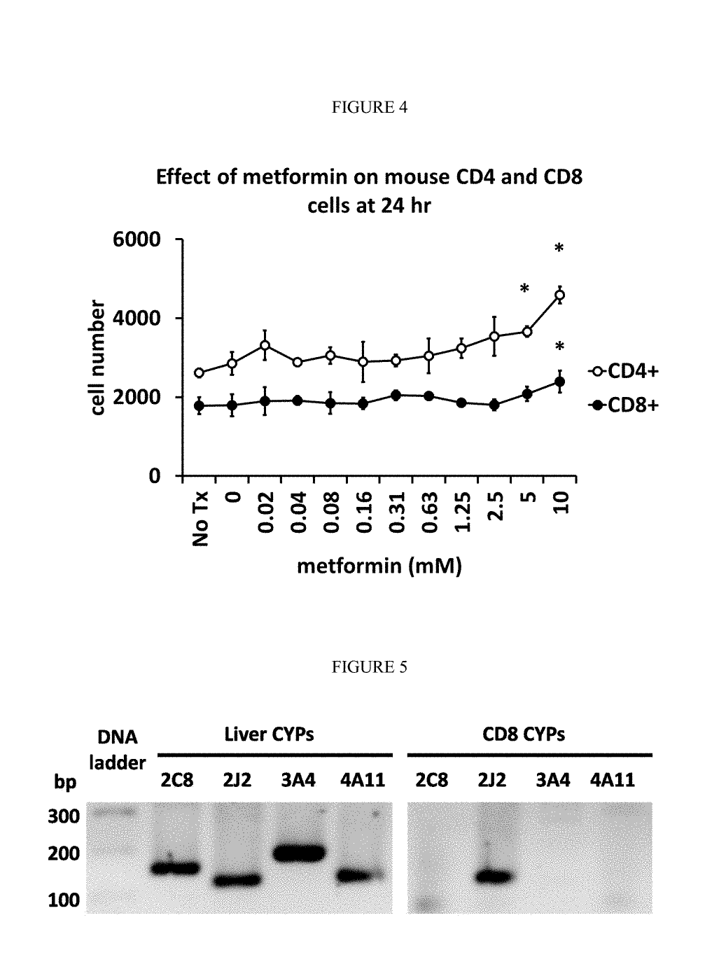

At low concentrations (0.09 to 1.5 uM) HBB did not inhibit T cell growth, but at higher concentrations (3 to 25 uM) HBB caused proliferation of CD4+ and CD8+ T lymphocytes with optimum concentrations of 6 to 12 uM (1.9-fold for CD4+ and 1.6-fold for CD8+; P<0.05 for both), conditions that inhibited breast cancer cells. Notably, CD4+FoxP3+ regulatory T cells were not increased by HBB. Metformin treatment caused an increase of CD4+ and CD8+ T lymphocytes only at concentrations >5 mM (see, FIG. 4). Importantly, it is not possible to attain these concentrations of metfomrin in the tissues of animals. The maximum tissue concentrations of metformin in rodents is about 500 microM, which is 10-fold lower than the threshold concentration required to promote growth of T cells in vitro. For expansion of CD8+ T cells ex vivo, HBB is 5-fold more effective at 1000-fold lower concentration than metformin (the increase of CD8+ T cells with metformin is 14% (10 mM) compared to 60% for HBB at 6 to 12 microM). For expansion of CD4+ T cells ex vivo, HBB is 3-fold more effective at 1000-fold lower concentration (the increase of CD4+ T cells with metformin is 30% at 10 mM concentration compared to 90% for HBB at 6 to 12 microM). Therefore, metformin is very unlikely to have proliferative activity on T cells at achievable concentrations, whereas HBB is effective because of its 1000-fold greater potency for T cell effects and 3 to 5-fold greater efficacy.

### Conclusions

HBB potently increases the proliferation of CD4+ and CD8+ mouse T lymphocytes stimulated with anti-CD3 and anti-CD28 antibodies at concentrations that suppress human and mouse mammary carcinoma cells. In contrast, metformin failed to show proliferation-inducing activity for T cells at concentrations below 5 mM. HBB therefore is a potent T lymphocyte activator, while inhibiting breast cancer epithelial cells. These results indicate that HBB may be effective for the treatment of cancer, as well as to facilitate immune therapy for a variety of diseases and disorders.

### Example 2. N1-hexyl-N5-benzyl-biguanide (HBB) Inhibits Regulatory T Cell Generation

As described below, the effects of HBB on two distinct populations of progenitor regulatory T cell populations, as well as naive T cells, were examined.

### Methods

### TRP Conversion Assays

Each TRP (CD4+CD25+FOXP3- or CD4+CD25−FOXP3+) population was isolated by FACS and stimulated with 1 U/mL IL2 (in complete RPMI) for 3 days then analyzed for upregulation of CD25 and FOXP3 (by GFP reporter).

iTreg Differentiation

Naive, non-Treg CD4 T cells were first isolated (these are isolated by magnetic depletion on CD8, CD11b, CD11c, CD19, CD24, CD25, CD44, CD49b, CD105, MHC-II, Gr1, Ter119, B220, TCRg/d). These cells were stimulated with plate bound anti-CD3/CD28 (plates were coated with 5 ug/mL for 2 hours at 37° C.) and given 100 U/mL IL2, 5 ng/mL TGFb and an additional 1 ug/mL anti-Cd28 in cRPMI. After 3 days the cells were stained for CD4, CD8 and CD25 with FOXP3 being on the GFP reporter. Tregs are defined here as CD25+FOXP3+. For this assay, 300,000 cells were plated per well in a 24 well plate.

### Results

As shown in FIGS. 6A-6C, HBB blocks the IL2-dependent conversion of both CD4+CD25+FOXP3- and CD4+CD25−FOXP3+ progenitor cells into Tregs.

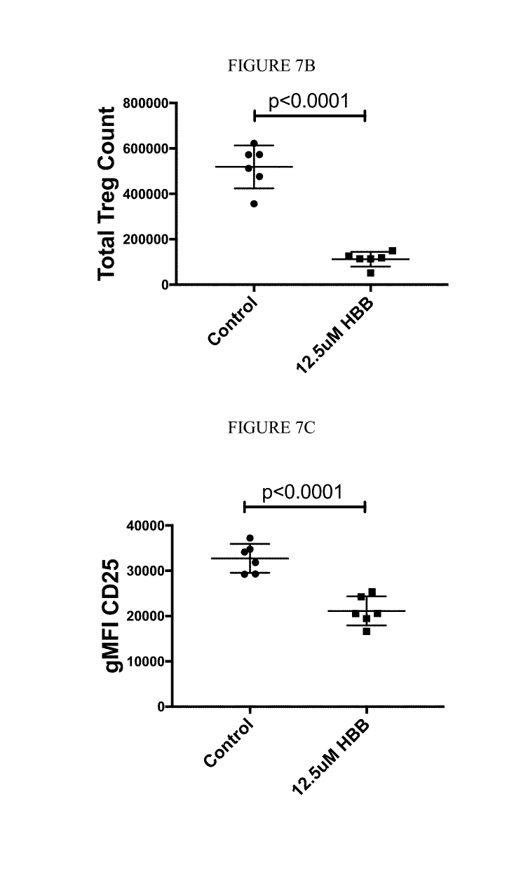

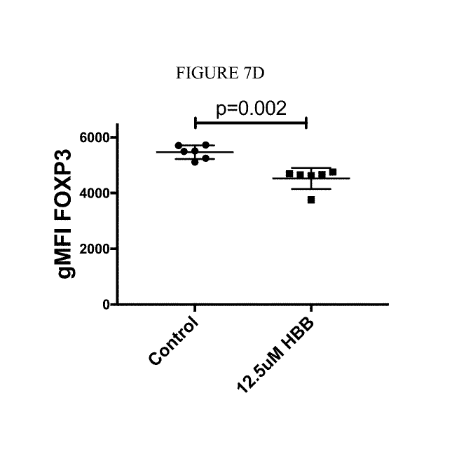

Additionally, HBB inhibits the differentiation of naive T cells into Tregs in the presence of IL2 and TGFb efficiently (FIGS. 7A-7D). The few Treg cells that were generated expressed less FOXP3 and CD25, and therefore, are likely to be poor suppressor cells.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A method of promoting T lymphocyte proliferation, comprising contacting a population of cells comprising T lymphocytes with an effective amount of N1-hexyl-N5-benzyl biguanide (HBB), or a pharmaceutically acceptable salt thereof, wherein contact with HBB, or a pharmaceutically acceptable salt thereof, causes proliferation of the T lymphocytes, and wherein the proliferated T lymphocytes are not regulatory T cells.

2. The method of claim 1, wherein the proliferated T lymphocytes are selected from the group consisting of effector T cells, T helper cells, cytotoxic T cells, adoptive T cells, memory T cells, natural killer T cells, mucosal associated invariant T cells, gamma delta T cells and chimeric antigen receptor T cells (CAR-T).

3. The method of claim 1, wherein the proliferated T lymphocytes express CYP2J2 and do not express CYP3A4.

4. The method of claim 1, wherein the number of T lymphocytes in the population of cells increases by at least about 20% after being contacted with HBB, or a pharmaceutically acceptable salt thereof.

5. The method of claim 1, wherein the population of cells comprising T lymphocytes is contacted with HBB, or a pharmaceutically acceptable salt thereof, in vivo.

6. The method of claim 1, wherein the population of cells comprising T lymphocytes is contacted with HBB, or a pharmaceutically acceptable salt thereof, in vitro or ex vivo.

7. The method of claim 1, further comprising activating the T lymphocytes by contacting the population of cells comprising the T lymphocytes with an anti-CD3 and/or anti-CD28 antibody.

8. The method of claim 1, further comprising administering the HBB-treated, proliferated T lymphocytes to a patient.

9. A method of promoting T lymphocyte proliferation in a patient in need thereof, comprising administering to the patient an effective amount of N1-hexyl-N5-benzyl biguanide (HBB), or a pharmaceutically acceptable salt thereof, wherein the proliferated T cells are not regulatory T cells.

10. The method of claim 9, wherein the patient has cancer.

11. The method of claim 10, wherein the cancer is selected from the group consisting of squamous cell cancer, small-cell lung cancer, non-small cell lung cancer, adenocarcinoma of the lung, squamous carcinoma of the lung, cancer of the peritoneum, hepatocellular cancer, renal cell carcinoma, gastrointestinal cancer, gastric cancer, esophageal cancer, pancreatic cancer, glioma, cervical cancer, ovarian cancer, liver cancer, bladder cancer, hepatoma, breast cancer (e.g., endocrine resistant breast cancer), colon cancer, rectal cancer, lung cancer, endometrial or uterine carcinoma, salivary gland carcinoma, kidney cancer, liver cancer, prostate cancer, vulval cancer, thyroid cancer, hepatic carcinoma, melanoma, leukemia and other lymphoproliferative disorders, and various types of head and neck cancer.

12. The method of claim 9, further comprising administering one or more additional therapeutic agents to the patient.

13. The method of claim 12, wherein the one or more additional therapeutic agents are selected from the group consisting of anti-CTLA4 antibodies, anti PD-1 antibodies and anti PD-L1 antibodies, or fragments thereof.

14. A method of inhibiting the generation of regulatory T cells in a patient in need thereof comprising administering to the patient an effective amount of N1-hexyl-N5-benzyl biguanide (HBB), or a pharmaceutically acceptable salt thereof.

15. The method of claim 14, wherein the method inhibits the conversion of progenitor regulatory T cells into regulatory T cells.

16. The method of claim 14, wherein the method inhibits the differentiation of naive T cells into regulatory T cells.

17. The method of claim 14, wherein the patient has cancer.

18. The method of claim 17, wherein the cancer is selected from the group consisting of squamous cell cancer, small-cell lung cancer, non-small cell lung cancer, adenocarcinoma of the lung, squamous carcinoma of the lung, cancer of the peritoneum, hepatocellular cancer, renal cell carcinoma, gastrointestinal cancer, gastric cancer, esophageal cancer, pancreatic cancer, glioma, cervical cancer, ovarian cancer, liver cancer, bladder cancer, hepatoma, breast cancer (e.g., endocrine resistant breast cancer), colon cancer, rectal cancer, lung cancer, endometrial or uterine carcinoma, salivary gland carcinoma, kidney cancer, liver cancer, prostate cancer, vulval cancer, thyroid cancer, hepatic carcinoma, melanoma, leukemia and other lymphoproliferative disorders, and various types of head and neck cancer.

19. A method for treating cancer in a patient comprising administering to the patient an effective amount of 1) N1-hexyl-N5-benzyl biguanide (HBB), or a pharmaceutically acceptable salt thereof; and 2) one or more immune checkpoint inhibitors, wherein the immune checkpoint inhibitors are selected from the group consisting of CTLA4, PD-1 and PD-L1 inhibitors.

20. The method of claim 19, wherein the CTLA4, PD-1 or PD-L1 inhibitor is selected from the group consisting of nivolumab, pembrolizumab, pidilizumab, AMP-224, MEDI0680, spartalizumab, atezolizumab, avelumab, durvalumab, BMS-936559, ipilimumab and tremelimumab.

21. A method of inhibiting mechanistic target of rapamycin (mTOR) and/or electron transfer (ETC) in one or more cells in a patient in need thereof, comprising administering to the patient an effective amount of N1-hexyl-N5-benzyl biguanide (HBB), or a pharmaceutically acceptable salt thereof.


SEQUENCE LISTING (8 sequences)

SEQ ID NO: 1 - DNA, 20 bp, Artificial Sequence
  Description of Artificial Sequence Synthetic primer
agatcagaattttctcaccc

SEQ ID NO: 2 - DNA, 20 bp, Artificial Sequence
  Description of Artificial Sequence Synthetic primer
aacttcgtgtaagagcaaca

SEQ ID NO: 3 - DNA, 20 bp, Artificial Sequence
  Description of Artificial Sequence Synthetic primer
gcccgggagtccatgcccta

SEQ ID NO: 4 - DNA, 20 bp, Artificial Sequence
  Description of Artificial Sequence Synthetic primer
gggcaggtggtacccagcca

SEQ ID NO: 5 - DNA, 20 bp, Artificial Sequence
  Description of Artificial Sequence Synthetic primer
gcctggtgctcctctatcta

SEQ ID NO: 6 - DNA, 21 bp, Artificial Sequence
  Description of Artificial Sequence Synthetic primer
ggctgttgaccatcataaaag

SEQ ID NO: 7 - DNA, 22 bp, Artificial Sequence
  Description of Artificial Sequence Synthetic primer
catggcagactctgtacgagtg

SEQ ID NO: 8 - DNA, 22 bp, Artificial Sequence
  Description of Artificial Sequence Synthetic primer
ctgatggctgaaggcacacttc